(19) United States
(12) Patent Application Publication
(10) Pub. No.: US 2011/0046512 A1
(43) Pub. Date: Feb. 24, 2011

(54) Flared Needle for EUS Fine Needle Aspiration Device

(76) Inventors: Daniel BACON, Fitchburg, MA (US); John Griego, Blackstone, MA (US); Michal Weisman, Fairlawn, NJ (US)

Correspondence Address: FAY KAPLUN & MARCIN, LLP, 150 BROADWAY, SUITE 702, NEW YORK, NY, 10038, US

(21) Appl. No.: 12/852,938

(22) Filed: Aug. 9, 2010

Related U.S. Application Data
(60) Provisional application No. 61/235,465, filed on Aug. 20, 2009.

(51) Int. Cl. A61B 10/02

(52) U.S. Cl. 600/566

(57) ABSTRACT

A device for gathering tissue samples includes a needle defining a distal tissue penetrating tip. The needle includes an increased diameter proximal portion extending proximally from the distal tip. An outer diameter of the distal tip is smaller than an outer diameter of the proximal portion. The needle defines a lumen extending therethrough from a distal opening in the distal tip thereof to a proximal opening at a proximal end thereof. The lumen includes a distal portion having a first inner diameter and a proximal portion having a second diameter greater than the first diameter. The device also includes a stylet slidably received within the lumen. An outer diameter of a distal portion of the stylet is substantially equal to the inner diameter of the distal portion of the lumen so that, when the stylet is in an extended position extending to the distal opening of the lumen, the lumen is substantially sealed thereby, and when, in a retracted position in which a distal end of the stylet is received within the proximal portion of the lumen, an annular space surrounding the stylet defines an aspiration path for samples exiting the distal portion of the lumen.

### PRIORITY CLAIM

This application claims the priority to the U.S. Provisional Application Ser. No. 61/235,465, entitled “Flared Needle for EUS Fine Needle Aspiration Device,” filed on Aug. 20, 2009.

### BACKGROUND

Biopsy procedures may be performed with Endoscopic Ultrasound Fine Needle Aspiration (“EUS-FNA”) devices to obtain cells or small samples of tissue from, for example, the breast or liver for cytology studies, endoscopy or oncology. As understood by those skilled in the art, biopsy needles enable the capture of samples to facilitate diagnosis and treatment. However, in many instances, EUS-FNA devices yield samples that are too small, which are contaminated during the biopsy procedure or which are otherwise flawed to the extent that a thorough analysis and diagnosis is not possible. In these cases resampling of the tissue is required involving multiple instances of removal and insertion of the stylet which, in turn, requires removing and replacing the syringe coupled to the needle. This increases the time and expense associated with the EUS-FNA procedure.

With current EUS-FNA devices, a stylet is inserted through the lumen to extend to the distal end thereof to prevent tissue from entering the needle as the needle is passed through tissue along a path toward a target site to be sampled. When the needle has reached the target site, the stylet is removed from the device to allow for the application of negative pressure therethrough to aspirate sample tissue into the distal end of the needle. After the sample has been obtained, the stylet may be re-inserted into the needle to push the sample out of the distal end of the needle (e.g., onto a slide or into another collection area). As described above, when an additional sample is required, the stylet must be repositioned at the distal end of the needle and the needle must then be re-inserted through the skin and intervening tissue to the target site.

### SUMMARY OF THE INVENTION

The present invention relates to a device for gathering tissue samples, comprising a needle defining a distal tissue penetrating tip, the needle including an increased diameter proximal portion extending proximally from the distal tip, an outer diameter of the distal tip being smaller than an outer diameter of the proximal portion, the needle defining a lumen extending therethrough from a distal opening in the distal tip thereof to a proximal opening at a proximal end thereof, the lumen including a distal portion having a first inner diameter and a proximal portion having a second diameter greater than the first diameter in combination with a stylet slidably received within the lumen, an outer diameter of a distal portion of the stylet being substantially equal to the inner diameter of the distal portion of the lumen so that, when the stylet is in an extended position extending to the distal opening of the lumen, the lumen is substantially sealed thereby, and when, in a retracted position in which a distal end of the stylet is received within the proximal portion of the lumen, an annular space surrounding the stylet defines an aspiration path for samples exiting the distal portion of the lumen.

### BRIEF DESCRIPTION OF THE DRAWINGS

FIG. 1 is a side view of a biopsy needle apparatus according to a first embodiment of the invention;

FIG. 2 is a partially cross-sectional view of the biopsy needle of FIG. 1;

FIG. 3 is a cross-sectional view of the biopsy needle of FIG. 1 in a first configuration;

FIG. 4 is a cross-sectional view of the biopsy needle of FIG. 1 in a second configuration;

FIG. 5 is a cross-sectional view of the biopsy needle according to a second embodiment of the invention;

FIG. 6 is a perspective view of a proximal portion of a biopsy needle according to a third embodiment of the invention; and

FIG. 7 is a partial cross-section view of the biopsy needle of FIG. 6.

### DETAILED DESCRIPTION

The present invention, which may be further understood with reference to the following description and the appended drawings, relates to an apparatus and method for obtaining tissue samples and, more particularly relates to EUS-FNA devices. The needle design of one embodiment of the present invention which may be used in substantially all procedures employing EUS-FNA devices further increases the efficacy of EUS-FNA procedures by permitting a retraction of a stylet so that, when retracted, a distal length of the stylet is separated from a distal end of a needle but remains within a proximal portion of the needle.

Devices and methods according to the present invention employ an FNA device comprising a stylet which seals an inner lumen of the needle during the initial penetration into body tissue (e.g., the stomach or the duodenal) wall to minimize the entry of blood and tissue into the needle before a target sampling site has been reached and which is retracted into the device during tissue sampling. It is noted that the use of the term distal herein refers to a direction away from a user and toward a target tissue treatment area and the term proximal refers to a direction approaching a user of the device (e.g., a physician) with a proximal portion of the device remaining external to the patient as the distal portion is inserted into the body.

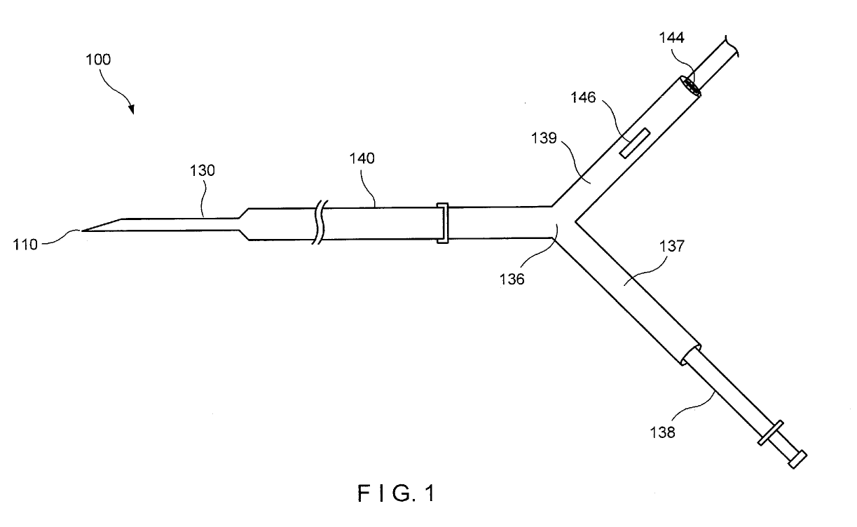

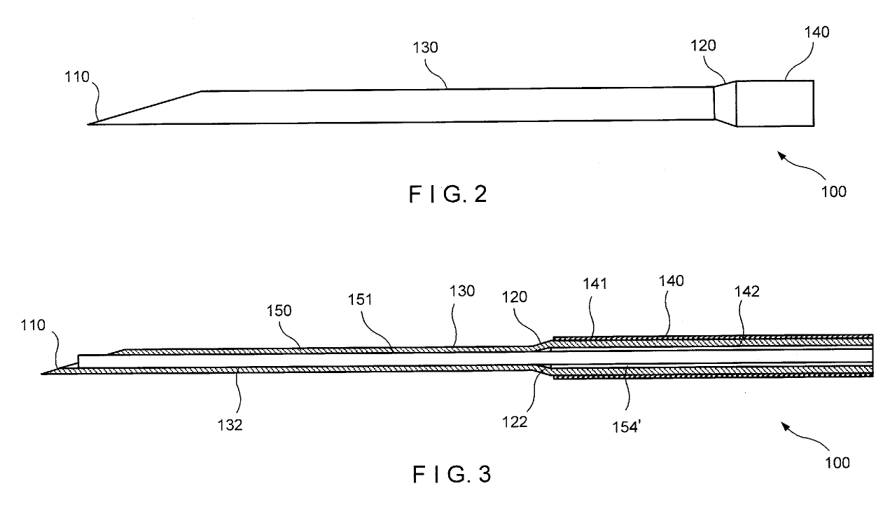

As shown in FIGS. 1-4, a device 100 according to an exemplary embodiment of the present invention for use with an EUS-FNA actuation mechanism (not shown) comprises an elongated hollow body 130 with a puncturing point 110 at a distal end thereof. As would be understood by those skilled in the art, the puncturing point 110 is preferably formed as an echosonic tip to facilitate viewing under ultrasound imaging as would be understood by those skilled in the art. Specifically, a portion of the body 130 adjacent to the puncturing point 110 may be formed with dimples, rings, or another pattern to create a greater reflective surface. The pattern may be formed by laser etching or another technique known in the art. The elongated body 130 which may be composed, for example, of stainless steel, nitinol or another suitable material, includes a lumen 132 extending therethrough to a distal opening 134 at the puncturing point 110. In a preferred embodiment, the elongated body 130 is formed of stainless steel due to the ease of machining, welding, and adhesively bonding this material. A flared portion 120 formed at the proximal end of the body 130 gradually transitions the outer diameter of the device 100 from the diameter of the body 130 to an increased diameter portion 141 extending proximally therefrom. In this embodiment, the increased diameter portion 141 is encased by an extrusion 140 positioned thereover and extending to a proximal end of the device 100. The flared portion 120 and increased diameter portion 141 may be integrally formed with the body 130 and the extrusion 140. In another embodiment of the present invention, as shown in FIG. 5, an extrusion 140′ may be formed as a separate element engaging an increased diameter portion 141′ with a friction fit or may be otherwise bonded thereto using any bonding means known in the art, as will be described in greater detail later.

The increased diameter portion 141 includes a lumen 142 extending therethrough. The lumen 142 is in fluid communication the lumen 132 extending through the body 130. The lumen 142 is also in communication with a handle 136 which remains outside the body during use. As seen in FIG. 3, an inner diameter of the lumen 132 is reduced with respect to an inner diameter of the lumen 142. Additionally, a ramp 122 formed within the flared portion 120 gradually transitions the inner diameter of the lumen 142 down to that of the lumen 132 to facilitate insertion of a stylet 150 into the lumen 132, as will be described below. Those skilled in the art will understand that this lumen diameter transition need not be made gradually through the use of a ramp but may take the form of a shoulder extending in a plane substantially perpendicular to an axis of the extrusion 140 with a rounded surface at the opening to the lumen 132 facilitating insertion of the stylet 150. The extrusion 140 may be extruded from a polymer or other suitable material such as PTFE (Teflon®), Nylon 12 or Pebax 7233, a coil of metal such as stainless steel, nitinol, or a polymer such as Polyetheretherketone (“PEEK”) or another suitable material sealed by a shrink wrap or other coating of material as would be understood by those skilled in the art.

The handle 136 according to this embodiment is substantially Y-shaped and includes a first leg 137 including a coupling for attaching a syringe 138 or other source of aspiration pressure thereto and a second leg 139 including a port 144 through which a stylet 150 may be inserted and withdrawn. Alternatively, the apparatus for applying aspiration pressure may be permanently coupled to the first leg 137. As would be understood by those skilled in the art, the port 144 may include a valve (not shown) to prevent aspiration pressure applied to the device 100 from leaking therefrom. The handle 136 also preferably includes an actuator 146 for advancing and retracting an outer sheath (not shown) formed over the elongated body 130. The outer sheath (not shown) protects the elongated body 130 during delivery to a target site in the body. Once the target site has been reached, manipulation of the actuator 146 causes a proximal retraction of the outer sheath to expose a predetermined distal portion of the elongated body 130.

As shown in FIG. 3, when in an extended configuration, the stylet 150 is positioned at a distal end of the needle 100 closing the distal opening 134 of the lumen 132 so that, as device 100 is advanced through tissue to a target tissue sampling site, unwanted material such as blood and/or tissue is prevented from entering and clogging the lumen 132. The stylet 150 is preferably sized to substantially seal the distal opening 134 and prevent the passage of foreign particles thereinto.

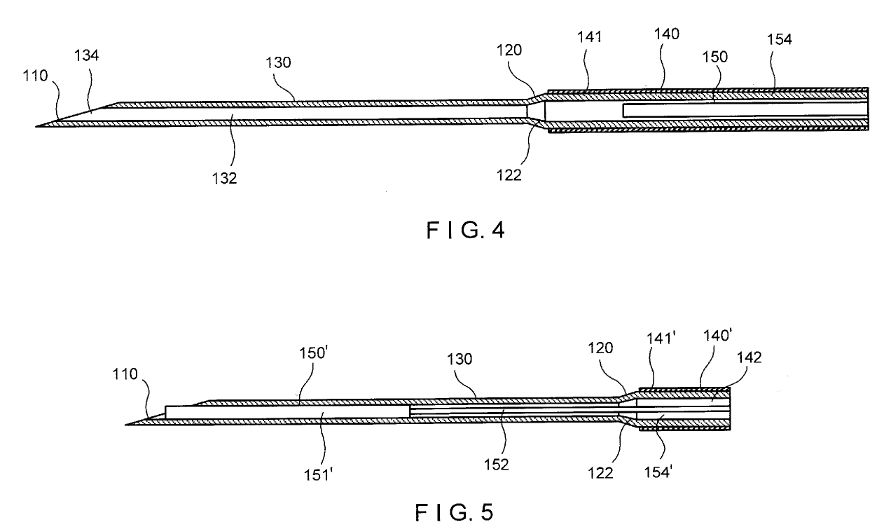

Once the needle 100 has been moved to the target sampling site, the stylet 150 is retracted into the lumen 142 as shown in FIG. 4. When retracted into the lumen 142, an annular space 154 remains around the portion 151 of the stylet 150 and an annular space 154′ around the proximal portion 152 which accounts for the majority of the length of the stylet 150 is even larger than the space 154. This facilitates aspiration of sampled tissue through the lumen 142 even when the stylet 150 remains therein as pressure and the sampled tissue may pass through this annular space 154. As the stylet 150 need not be withdrawn from the device 100 to aspirate samples through the lumen 142, the lumen 132 may be cleared without removing the syringe 138 and additional samples may be collected in less time and with less trauma to the patient.

Furthermore, the device 100 according to the present invention retains enhanced flexibility as the diameter of the body 130 is substantially the same as that of similar biopsy needles. Non-flared needles on the other hand, require a greater overall diameter to allow for aspiration and this reduces the flexibility and, consequently, the ability of the needle to traverse tortuous paths while increasing deformation of the needle which, in turn reduces visibility under ultrasound procedures.

As described earlier, a first alternate embodiment of the present invention, as shown in FIG. 5, may comprise an extrusion or sheath 140′ configured to be slidably received over the increased diameter portion 141′. The increased diameter portion 141′ of this embodiment extends proximally from the flared portion 120 by a predetermined distance and is formed substantially similarly as the increased diameter portion 141 of FIGS. 1-4. An exemplary stylet 150′ employed with the device of FIG. 5 may comprise a distal portion 151′ with a diameter sized to substantially fill the lumen 132 and a proximal portion 152 having a substantially reduced diameter to minimize the portion of the lumen 142 occupied by the stylet 150, as those skilled in the art will understand.

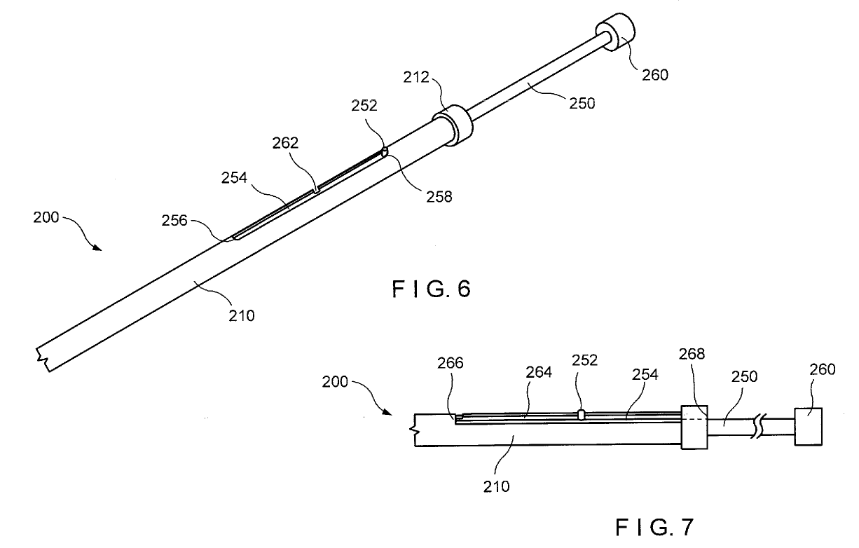

It is noted that, although the present invention has been disclosed with respect to FNA biopsy procedures, various alternate applications may be employed. For example, the present invention may be employed in biliary and pulmonary procedures. Those skilled in the art will understand that such procedures often require the use of a stylet that is 270 centimeters in length or longer wherein removing and replacing the long stylet is time consuming. The exemplary design of the present invention overcomes this issue by eliminating the need to extract the stylet from the device for aspiration. Rather, the stylet is simply retracted into the lumen, proximal to the flared portion. To aid in the partial retraction of the stylet, a track may be provided in a handle of the device. Specifically, as shown in FIGS. 6-7, a device 200 formed substantially similarly as the device 100 of FIGS. 1-4 comprises an elongated body 210 extending to a proximal end 212 and having a stylet 250 insertable therethrough. The stylet 250 is provided with a stop 252 configured to engage a track 254 extending along a predetermined length of the elongated body 210. The stop 252 is configured to permit sliding of the stylet 250 relative to the elongated body 210 to one of a fully advanced position when the stop 254 is advanced to a distal end 256 of the track 254 and a fully retracted position when the stop 254 is retracted to a proximal end 258 of the track 254. The exemplary track 254 of the present invention is configured to permit a proximal end 260 of the stylet 250 to be retracted out of the elongated body 210 by only the distance necessary to permit aspiration, as those skilled in the art will understand. The length and position of the track 254 may therefore be modified to conform to the requirements of the procedure being performed without deviating from the scope of the present invention. If the stylet 250 is to be completely removed from the elongated body 210, the stop 252 may be moved laterally to pass through a channel 262 into a removal track 264 that extends parallel to the track 254 and is further open to a proximal end of the elongated body 210. Specifically, the removal track 264 may be formed substantially similarly as the track 254 and may extend from a distal end 266 to a proximal end 268 that permits disengagement of the stop 252 therewith. It is further noted that a position of the channel 262 connecting the track 254 to the removal track 264 may be modified to any suitable location without deviating from the scope of the present invention.

It is further submitted that the feature of each of the aforementioned embodiments of the present invention may be combined without deviating from the spirit and scope of the present invention. The present invention has been described with reference to specific exemplary embodiments. Those skilled in the art will understand that various modifications and changes may be made to the embodiments. The specifications are, therefore, to be regarded in an illustrative rather than a restrictive sense.

What is claimed is:

1. A device for gathering tissue samples, comprising:
a needle defining a distal tissue penetrating tip, the needle including an increased diameter proximal portion extending proximally from the distal tip, an outer diameter of the distal tip being smaller than an outer diameter of the proximal portion, the needle defining a lumen extending therethrough from a distal opening in the distal tip thereof to a proximal opening at a proximal end thereof, the lumen including a distal portion having a first inner diameter and a proximal portion having a second diameter greater than the first diameter; and
a stylet slidably received within the lumen, an outer diameter of a distal portion of the stylet being substantially equal to the inner diameter of the distal portion of the lumen so that, when the stylet is in an extended position extending to the distal opening of the lumen, the lumen is substantially sealed thereby, and when, in a retracted position in which a distal end of the stylet is received within the proximal portion of the lumen, an annular space surrounding the stylet defines an aspiration path for samples exiting the distal portion of the lumen.

2. The device according to claim 1, further comprising:
a flared portion connecting the proximal portion and the distal tip of the needle, an outer diameter of the flared portion gradually increasing from a distal end thereof adjacent to a proximal end of the distal tip to a proximal end at a distal end of the proximal portion.

3. The device according to claim 1, further comprising:
an extrusion extending over a proximal portion of the needle.

4. The device according to claim 2, wherein the flared portion is integrally formed with the needle.

5. The device according to claim 2, wherein the extrusion is attached to the needle by one of a friction fit and welding.

6. The device according to claim 1, wherein a diameter of the stylet is substantially uniform along its length.

7. The device according to claim 1, wherein the inner diameter of the distal portion of the lumen is substantially uniform.

8. The device according to claim 1, wherein a diameter of a proximal portion of the stylet is less than the diameter of the distal portion of the stylet.

9. The device according to claim 1, wherein the needle is formed of one of stainless steel and nitinol.

10. The device according to claim 1, wherein the extrusion includes one of an extruded structure and a coil sealed by one of a coating and a heat shrink material.

11. The device according to claim 1, further comprising:
a handle including a coupling for attaching a source of aspiration pressure to the lumen and an actuator for moving the stylet between the extended and retracted positions.

12. The device according to claim 1, further comprising:
a first track extending along a predetermined length of extrusion and configured to engage an abutment extending out of the stylet, engagement of the first track with the abutment guiding proximal and distal movement of the stylet from the extended position to the retracted position.

13. The device according to claim 12, further comprising:
a second track extending parallel to the first track and being connected thereto by a channel extending therebetween, wherein a proximal end of the second track remains open to a proximal end of the extrusion to permit disengagement of the abutment therefrom.

14. The device according to claim 1, further comprising:
a first track extending along a predetermined length of the needle and configured to engage an abutment extending out of the stylet, engagement of the first track with the abutment guiding proximal and distal movement of the stylet from the extended position to the retracted position.

15. The device according to claim 14, further comprising:
a second track extending parallel to the first track and being connected thereto by a channel extending therebetween, wherein a proximal end of the second track remains open to a proximal end of the flexible sheath to permit disengagement of the abutment therefrom.

16. The device of claim 1, wherein the distal portion of the lumen resides within the distal portion of the needle and the proximal portion of the needle resides in the proximal portion of the needle.

17. A biopsy needle, comprising:
an elongate member including a distal portion extending from a tissue penetrating distal tip of the elongate member to a proximal end and a proximal portion extending proximally from the proximal end of the distal portion to a proximal end of the elongate member, an outer diameter of the distal portion being smaller than a diameter of the proximal portion, the needle including a lumen extending therethrough from a distal opening in the distal tip to a proximal opening at the proximal end of the elongate member, an inner diameter of the distal part of lumen extending through the distal portion having an inner diameter smaller than an inner diameter of a proximal portion of the lumen extending through the proximal portion of the needle;
a stylet slidably received within the lumen, an outer diameter of a distal portion of the stylet being substantially equal to the inner diameter of the distal portion of the lumen so that, when the stylet is in an extended position extending to the distal opening of the lumen, the lumen is substantially sealed thereby, and when, in a retracted position in which a distal end of the stylet is received within the proximal portion of the lumen, an annular space surrounding the stylet defines an aspiration path for samples exiting the distal portion of the lumen; and
a handle coupled to the proximal end of the elongate member.

18. The device according to claim 17, wherein the handle includes an actuator for moving the stylet between the extended and retracted positions.
